SEQUENCE LISTING (8 sequences)

SEQ ID NO: 1 - DNA, 3135 bp, Artificial Sequence
  betaKlotho CDS - codon optimized
atgaagcctggatgtgccgccggaagccccggcaacgagtggatcttcttcagcaccgacgagatcaccacccggtacagaaacaccatgagcaacggcggcctgcagcggagcgtgatcctgtctgctctgatcctgctgagagccgtgaccggctttagcggcgacggcagagccatctggtccaagaaccccaacttcacccccgtgaacgagagccagctgttcctgtacgataccttccccaagaacttcttctggggcatcggcacaggcgccctgcaggtggaaggcagctggaagaaggatggcaagggccccagcatctgggaccacttcatccacacccacctgaagaacgtgtccagcaccaacggcagcagcgacagctacatcttcctggaaaaggacctgagcgccctggacttcatcggcgtgtccttctaccagttcagcatcagctggcccagactgttccccgacggcatcgtgacagtggccaatgccaagggcctgcagtactacagcaccctgctggacgccctggtgctgagaaacatcgagcccatcgtgaccctgtaccactgggacctgccactggctctgcaggagaaatacggcggctggaaaaacgacaccatcatcgacatcttcaacgactacgccacctactgcttccagatgttcggcgacagagtgaagtactggatcaccatccacaacccctacctggtggcctggcacggctatggcaccggaatgcatgcccctggcgagaagggaaatctggccgccgtgtacaccgtgggccacaacctgatcaaggcccacagcaaagtgtggcacaactacaacacccacttccggccccaccagaagggctggctgtctatcacactgggcagccactggatcgagcctaaccgcagcgagaacaccatggatatcttcaagtgccagcagagcatggtgtccgtgctgggatggttcgccaaccccattcatggcgacggcgactaccccgagggcatgcggaagaaactgttcagcgtgctgcccatcttcagcgaggccgagaagcacgagatgcggggcaccgccgatttcttcgcctttagcttcggccccaacaacttcaagcccctgaataccatggccaagatgggccagaatgtgtccctgaacctgagagaggccctgaactggatcaagctggagtacaacaacccccggatcctgatcgccgagaacggctggttcaccgacagcagagtgaaaaccgaggacaccaccgccatctatatgatgaagaacttcctgagccaggtgctgcaggctatccggctggatgagatccgggtgttcggctacacagcctggtcactgctggatggcttcgagtggcaggacgcctacaccatcagacggggcctgttctacgtggacttcaacagcaagcagaaagagcggaagcccaagagcagcgcccactactacaagcagatcatcagagagaatggcttcagcctgaaagaaagcacccccgacgtgcagggccagttcccctgtgatttttcctggggcgtgaccgagagcgtgctgaagcctgagtctgtggcctccagcccccagtttagcgacccccacctgtacgtgtggaacgccaccggcaaccggctgctgcatagagtggaaggcgtgcggctgaaaaccagacccgcccagtgcaccgacttcgtgaacatcaagaaacagctggaaatgctggcccggatgaaagtgacccactacagattcgccctggattgggcctccgtgctgcctaccggaaacctgtctgccgtgaacagacaggccctgaggtactacagatgcgtggtgtccgagggcctgaagctgggcatcagcgccatggtcacactgtactaccccacccacgcccacctgggactgcctgaacctctgctgcatgccgacggctggctgaatccttctacagccgaggccttccaggcctacgccggactgtgctttcaggaactgggcgacctcgtgaagctgtggatcacaatcaacgagcccaacagactgagcgacatctacaacagaagcggcaacgacacctacggcgctgcccacaatctgctggtggctcatgccctggcctggcggctgtacgacagacagttcagaccaagccagcggggagccgtgtctctgtctctgcatgctgattgggccgagcccgccaacccttacgccgactctcattggagagccgccgagcggttcctgcagttcgagatcgcttggtttgccgagcccctgttcaagaccggcgattaccctgccgccatgagagagtatatcgccagcaagcacagaagaggcctgagcagctccgccctgcctagactgacagaggccgaaaggcggctgctgaagggcaccgtggatttctgtgccctgaatcacttcaccaccagattcgtgatgcacgagcagctggccggcagcagatacgacagcgaccgggacatccagtttctgcaggacatcacccggctgagcagccctacaagactggccgtgatcccttggggcgtgcggaagctgctgagatgggtgcgcagaaactacggcgacatggacatctatatcaccgccagcggcatcgacgaccaggccctggaagatgaccggctgcggaagtactacctgggcaagtacctgcaggaagtgctgaaggcctacctgatcgacaaagtgcggatcaagggctactacgccttcaagctggccgaggaaaagagcaagcccagattcggcttcttcaccagcgacttcaaggccaagtccagcatccagttctacaacaaagtgatcagcagcaggggcttcccattcgagaacagcagcagcagatgcagccagacacaggaaaacaccgagtgtaccgtgtgcctgttcctggtgcagaagaagcctctgatctttctgggctgctgcttctttagcacactggtgctgctgctgagcattgccatctttcagcggcagaagcggagaaagttttggaaggccaagaatctgcagcacatccccctgaaaaagggcaagcgggtggtgtcttga

SEQ ID NO: 2 - DNA, 5276 bp, Artificial Sequence
  [ptk-nl1.3 blas] ligation of ptk-nl1.3-neo.xdna [4208 nt]  (sali[3262] / kpn2i[2356]) to pcr brs-p(a)betaglo to ptknl.xdna [1060 nt]  (kpn2i[11] / sali[1075]) - 5276 bp
tctcctaattccttaatttaatacttattctgataatgataactaaaagatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaatgaagaccttcaacatctctcagcaggatctggagctggtggaggtcgccactgagaagatcaccatgctctatgaggacaacaagcaccatgtcggggcggccatcaggaccaagactggggagatcatctctgctgtccacattgaggcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgcagagagctcatctctgactatgctcctgactgctttgtgctcattgagatgaatggcaagctggtcaaaaccaccattgaggaactcatccccctcaagtacaccaggaactaaagctagaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttagaattccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggcgtcgacctgcagccaagcttatcgataaaataaaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 3 - DNA, 7109 bp, Artificial Sequence
  [pB-GAL4-Elk1a-PEST zeo ] Ligation of piggyBac carrier [5285 nt]  (AccI[2903] / BamHI[1737]) to PCR GAL4Elk1a-PEST zeo [1818 nt]  (BamHI[11] / AccI[1832]) - 7109 bp
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattcgaatttaaatcggatccgttaacgaattcgccaccatgaagctgctgagtagtattgaacaagcgtgtgacatatgccgacttaagaagctgaagtgcagcaaggaaaagccgaagtgcgccaaatgtcttaagaacaattgggagtgccggtactcgccaaagacgaagcgaagccctttgacacgcgctcatttgactgaagtggagagtcgcctggagcgccttgagcagttgttcctcctcatttttccccgtgaagacctcgacatgatactgaagatggatagtctccaagatattaaggccctgctcactggcctgtttgttcaggataacgtcaacaaggatgccgtgacggaccgccttgcctcggtggagacagatatgccacttaccttgcggcagcatcgcatctcggctaccatctcccagccgcagaagggccggaagccccgggacctagagcttccactcagcccgagcctgctaggtgggccgggacccgaacggaccccaggatcgggaagtggctccggcctccaggctccggggccggcgctgaccccatccctgcttcctacgcatacattgaccccggtgctgctgacacccagctcgctgcctcctagcattcacttctggagcaccctgagtcccattgcgccccgtagcccggccaagctctccttccagtttccatccagtggcagcgcccaggtgcacatcccttctatcagcgtggatggcctctcgacccccgtggtgctctccccagggccctctcacggctttccgcctgaggttgaagagcaagccgccggtacattgcctatgtcctgcgcacaagaaagcggtatggaccggcacccagccgcttgtgcttcagctcgcatcaacgtctaagcttccagaagccatgagaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaatgcatcggtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 4 - DNA, 9336 bp, Artificial Sequence
  [pB-hFGFR1c puro] Ligation of  piggyBac carrier [6093 nt]  (NheI[1714] / SpeI[1357]) to PCR hFGFR to pB.xdna [3235 nt]  (SpeI[11] / NheI[3250]) - 9336 bp
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtgtgccacctgacgtctaagaaaccattattatcatgacattaacctataaaaataggcgtagtacgaggccctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctagatcttcgaagaacaagtttgtacaaaaaagcaggcttggaaggagttcgaaccatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctagaattcgaattcgctagcgaattcgaatttaaatcggatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 5 - DNA, 9601 bp, Artificial Sequence
  [pB-KLB_OPTI puro] Ligation of  piggyBac carrier [6450 nt]  (NheI[1714] / NheI[1714]) to PCR KLB Opti to pB.xdna [3143 nt]  (NheI[11] / NheI[3158]) - 9601 bp
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgccaccatgaagcctggatgtgccgccggaagccccggcaacgagtggatcttcttcagcaccgacgagatcaccacccggtacagaaacaccatgagcaacggcggcctgcagcggagcgtgatcctgtctgctctgatcctgctgagagccgtgaccggctttagcggcgacggcagagccatctggtccaagaaccccaacttcacccccgtgaacgagagccagctgttcctgtacgataccttccccaagaacttcttctggggcatcggcacaggcgccctgcaggtggaaggcagctggaagaaggatggcaagggccccagcatctgggaccacttcatccacacccacctgaagaacgtgtccagcaccaacggcagcagcgacagctacatcttcctggaaaaggacctgagcgccctggacttcatcggcgtgtccttctaccagttcagcatcagctggcccagactgttccccgacggcatcgtgacagtggccaatgccaagggcctgcagtactacagcaccctgctggacgccctggtgctgagaaacatcgagcccatcgtgaccctgtaccactgggacctgccactggctctgcaggagaaatacggcggctggaaaaacgacaccatcatcgacatcttcaacgactacgccacctactgcttccagatgttcggcgacagagtgaagtactggatcaccatccacaacccctacctggtggcctggcacggctatggcaccggaatgcatgcccctggcgagaagggaaatctggccgccgtgtacaccgtgggccacaacctgatcaaggcccacagcaaagtgtggcacaactacaacacccacttccggccccaccagaagggctggctgtctatcacactgggcagccactggatcgagcctaaccgcagcgagaacaccatggatatcttcaagtgccagcagagcatggtgtccgtgctgggatggttcgccaaccccattcatggcgacggcgactaccccgagggcatgcggaagaaactgttcagcgtgctgcccatcttcagcgaggccgagaagcacgagatgcggggcaccgccgatttcttcgcctttagcttcggccccaacaacttcaagcccctgaataccatggccaagatgggccagaatgtgtccctgaacctgagagaggccctgaactggatcaagctggagtacaacaacccccggatcctgatcgccgagaacggctggttcaccgacagcagagtgaaaaccgaggacaccaccgccatctatatgatgaagaacttcctgagccaggtgctgcaggctatccggctggatgagatccgggtgttcggctacacagcctggtcactgctggatggcttcgagtggcaggacgcctacaccatcagacggggcctgttctacgtggacttcaacagcaagcagaaagagcggaagcccaagagcagcgcccactactacaagcagatcatcagagagaatggcttcagcctgaaagaaagcacccccgacgtgcagggccagttcccctgtgatttttcctggggcgtgaccgagagcgtgctgaagcctgagtctgtggcctccagcccccagtttagcgacccccacctgtacgtgtggaacgccaccggcaaccggctgctgcatagagtggaaggcgtgcggctgaaaaccagacccgcccagtgcaccgacttcgtgaacatcaagaaacagctggaaatgctggcccggatgaaagtgacccactacagattcgccctggattgggcctccgtgctgcctaccggaaacctgtctgccgtgaacagacaggccctgaggtactacagatgcgtggtgtccgagggcctgaagctgggcatcagcgccatggtcacactgtactaccccacccacgcccacctgggactgcctgaacctctgctgcatgccgacggctggctgaatccttctacagccgaggccttccaggcctacgccggactgtgctttcaggaactgggcgacctcgtgaagctgtggatcacaatcaacgagcccaacagactgagcgacatctacaacagaagcggcaacgacacctacggcgctgcccacaatctgctggtggctcatgccctggcctggcggctgtacgacagacagttcagaccaagccagcggggagccgtgtctctgtctctgcatgctgattgggccgagcccgccaacccttacgccgactctcattggagagccgccgagcggttcctgcagttcgagatcgcttggtttgccgagcccctgttcaagaccggcgattaccctgccgccatgagagagtatatcgccagcaagcacagaagaggcctgagcagctccgccctgcctagactgacagaggccgaaaggcggctgctgaagggcaccgtggatttctgtgccctgaatcacttcaccaccagattcgtgatgcacgagcagctggccggcagcagatacgacagcgaccgggacatccagtttctgcaggacatcacccggctgagcagccctacaagactggccgtgatcccttggggcgtgcggaagctgctgagatgggtgcgcagaaactacggcgacatggacatctatatcaccgccagcggcatcgacgaccaggccctggaagatgaccggctgcggaagtactacctgggcaagtacctgcaggaagtgctgaaggcctacctgatcgacaaagtgcggatcaagggctactacgccttcaagctggccgaggaaaagagcaagcccagattcggcttcttcaccagcgacttcaaggccaagtccagcatccagttctacaacaaagtgatcagcagcaggggcttcccattcgagaacagcagcagcagatgcagccagacacaggaaaacaccgagtgtaccgtgtgcctgttcctggtgcagaagaagcctctgatctttctgggctgctgcttctttagcacactggtgctgctgctgagcattgccatctttcagcggcagaagcggagaaagttttggaaggccaagaatctgcagcacatccccctgaaaaagggcaagcgggtggtgtcttgagctagcgaattcgaatttaaatcggatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 6 - DNA, 8582 bp, Artificial Sequence
  [pB-UAS-Luc hygro] Ligation of  piggyBac carrier [4905 nt]  (AccI[2903] / SpeI[1357]) to PCR UAS-Luc hygro to pB.xdna [3671 nt]  (SpeI[12] / AccI[3686]) - 8582 bp
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtggcctaactggccggtacccggaggactgtcctccgagtccggaggactgtcctccgagtccggaggactgtcctccgagtccggaggactgtcctccgagtccggaggactgtcctccgctcgaggatatcaagatctggcctcggcggccaagcttagacactagagggtatataatggaagctcgacttccagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactgtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 7 - DNA, 5459 bp, Artificial Sequence
  [pRL-TK(neo)] Ligation of  pRL-TK [4088 nt] (BamHI[2270] / BamHI[2270]) to PCR(MSCVneo) [1363 nt] (BamHI[11] / BamHI[1378])- 5459 bp
tctcctaattccttaatttaatacttattctgataatgataactaaaagatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgacctgcagccaagcttatcgataaaataaaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 8 - DNA, 88 bp, Homo sapiens
caaccggaagtggaacaaccggaagtggaacaaccggaagtggaacaaccggaagtggaacaaccggaagtggaacaaccggaagtgc

(19) United States
(12) Patent Application Publication
(10) Pub. No.: US 2024/0076332 A1
(43) Pub. Date: Mar. 7, 2024

(54) FGF21 Responsive Reporter Gene Cell Line

(71) Applicant: SVAR LIFE SCIENCE AB, Malmö (SE)

(72) Inventors: Michael TOVEY, PARIS (FR); Lue HUANG, PARIS (FR); Christophe LALLEMAND, PARIS (FR)

(21) Appl. No.: 16/339,059

(22) PCT Filed: Mar. 10, 2017
(86) PCT No.: PCT/EP2017/075055

§ 371 Date: Apr. 3, 2019

(30) Foreign Application Priority Data
Oct. 4, 2016 (EP) 16192296.8

(51) Int. Cl. C07K 14/50; C12N 5/071; C12N 15/85; G01N 33/68; G01N 33/74

(52) U.S. Cl. CPC C07K 14/50; C12N 5/0686; C12N 15/85; G01N 33/6854; G01N 33/74; C12N 2503/00; G01N 2333/50

(57) ABSTRACT

The present invention relates to a mammalian FGF21 responsive reporter cell line and method of using the same for detecting and optionally quantitating FGF21 activity in a test sample. The invention further relates to a method for detecting and optionally quantitating neutralizing antibodies against FGF21 present in a test sample using the reporter cell line of the present invention.

### CROSS-REFERENCE TO RELATED APPLICATIONS

This application is a national phase application under 35 U.S.C. § 371 of PCT International Application No. PCT/EP2017/075055, filed Mar. 10, 2017, which claims priority to European Application No. 16192296.8, filed Oct. 4, 2016, which are hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 65,678 Byte ASCII (Text) file named “38524-251_ST25,” created on Apr. 29, 2021.

### FIELD OF THE INVENTION

The present invention relates to a mammalian FGF21 responsive reporter cell line and method of using the same for detecting and optionally quantitating FGF21 activity in a test sample. The invention further relates to a method for detecting and optionally quantitating neutralizing antibodies against FGF21 present in a test sample using the reporter cell line of the present invention.

### BACKGROUND OF THE INVENTION

Human fibroblast growth factor 21 (FGF21) is a member of the family of the atypical fibroblast growth factors that include FGF19 and FGF23 in man. FGF21 stimulates glucose uptake in adipocytes and FGF21 is specifically induced by HMGCS2 (3-hydroxy-3-methylglutaryl-CoA synthase) to provide lipid-derived energy during carbohydrate deprivation during fasting. FGF21 has the potential to improve glycemic control in patients with type 2 diabetes, as well as the control of weight gain (W Y and Leung, 2016; Kliewer and Mangelsdorf, 2010; Fisher et al.).

Analogues of FGF21 are currently being tested in clinical trials in patients with type 2 diabetes and there is a need for a sensitive and specific assay both for the quantification of the potency and neutralizing antibody response to FGF21 and related analogues. FGF21 signals through a heterodimeric cell surface receptor composed of the tyrosine kinase FGFR1c receptor and beta-Klotho, single-pass membrane spanning protein with two glycosidase domains that is essential for optimal binding and activation of the FGF21 signal transduction pathway (Wang and Sun, 2009). Binding of FGF21 to the FGFR1c receptor in the presence of the beta-Klotho co-receptor leads to activation of the ras/raf MAP kinase signaling cascade, activation of ERK1/2 and phosphorylation and activation of the Elk-1 and SRF (Wang et al, 1997). Numerous other growth factors and cytokines present in human serum also employ the same signal transduction pathway rendering the development of a sensitive and specific assay for FGF21 difficult.

It follows that there is a need for a FGF21 responsive reporter gene cell line with for the detection of FGF21 activity while exhibiting minimal interference from normal human serum.

### SUMMARY OF THE INVENTION

The present invention was made in view of the prior art described above, and the object of the present invention is to provide a mammalian FGF21 responsive reporter cell line. In particular the object is to provide a mammalian FGF21 responsive reporter cell line, which is sensitive to presence of FGF21 activity and uses the same signal transduction pathway as FGF21 and without interference from other growth factors present in human serum that use the same signal transduction pathway as FGF21.

To solve the problem, the present invention provides a mammalian cell line comprising
(i) a first heterologous polynucleotide comprising a heterologous cis-acting regulatory sequence operably linked to a downstream promoter sequence, wherein said promoter is operably linked to an open reading frame encoding a first reporter protein,
(ii) a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and
(iii) a cell surface bound heterodimeric receptor protein comprising a tyrosine kinase FGFR1c and a beta-Klotho protein.

Another aspect of the present invention provides a method for detecting and optionally quantitating FGF21 activity in a test sample, said method comprising the steps of
(i) providing a test sample,
(ii) contacting said test sample with the cell line according to the present invention,
(iii) determining the activity of the first reporter protein in said cell line.

A further aspect of the present invention concerns a method for detecting and optionally quantitating neutralizing antibodies against FGF21 present in a test sample, said method comprising the steps of
(i) providing a test sample comprising,
(ii) providing a first and a second cell sample, wherein said cells samples comprises the cell line according to the present invention,
(iii) contacting the first cell sample with the test sample and subsequently with FGF21,
(iv) contacting the second cell sample with FGF21,
(iv) determining the activity of the first reporter protein in the cells of the first cell sample and determining the activity of the first reporter protein in the cells of the second cell sample,
(v) provide the ratio between the reporter activity in first and a second cell sample, where a ratio (first/second) lower than one is indicative for the presence of antibodies against FGF21 in said sample.

Consequently, present invention provides a cell line, the use thereof in various contexts allowing for the detection of FGF21 activity while exhibiting minimal interference from normal human serum or other growth factor present in human serum that use the same signal transduction pathway as FGF21. As a further consequence thereof, present invention in all its aspects does not require any sample manipulation in order to minimise or remove other growth factors (other than FGF21) using the same signal transduction pathway as FGF21 in order to provide a reliable result.

Moreover, in addition to providing a means of reducing the non-specific increase in the firefly-luciferase reporter-gene activity caused by the presence of growth factors such as native Elk-1 present in normal serum as discussed above, the invention also increases the sensitivity of detection of human FGF-21 as illustrated in Table 1. Thus, cells transfected according to the invention provides for increased sensitivity of detection of human FGF-21.

Thus, in one embodiment the invention provides a cell allowing for detection of FGF21 activity while exhibiting minimal interference from normal human serum or other growth factor present in human serum that use the same signal transduction pathway as FGF21.

In another embodiment, the invention provides a cell line with increased sensitivity of detection of human FGF-21 in comparison with other cell lines used in the art.

In yet a further embodiment, the invention provides a cell line which allows for detection of FGF21 activity while exhibiting minimal interference from normal human serum or other growth factor present in human serum that use the same signal transduction pathway as FGF21 and at the same time offers increased sensitivity of detection of human FGF-21 in comparison with other cell lines used in the art.

For example, HEK293 cells transfected with the native β-klotho gene exhibited an EC50 of approximately 24 ng/ml for FGF-21 whereas HEK293 cells transfected with the codon optimized β-klotho gene exhibited an EC50 of approximately 7.0 ng/ml for FGF-21 reflecting the increased sensitivity of the cell containing the codon optimized β-klotho gene. Furthermore, Jurkat cells transfected with the native β-klotho gene exhibited an EC50 of 172 ng/ml for FGF-21 (Table 1).

Consequently, in one aspect, the invention relates to a cell enabling detection of FGF-21 with a higher sensitivity, such that the EC50 of cells transfected according to the invention is at least decreased in comparison with cells transfected with the corresponding native gene or genes. The gene may be e.g. the β-klotho gene but may otherwise comprise other or further genes. The decrease may be about 2-fold, such as e.g. about 3-fold, such as e.g. about 4-fold, such as e.g. about 5-fold, such as e.g. 6-fold, such as e.g. 7-fold, such as e.g. about 8-fold, such as e.g. 9-fold, such as e.g. 10-fold, such as e.g. 50-fold, such as about 100-fold, or such as about 1000-fold.

### BRIEF DESCRIPTION OF THE DRAWINGS

FIG. 1 illustrates the structure of the Chimeric Elk-1-Gal4 Transcription Factor

FIG. 2 illustrates FGF21 Responsive Reporter-Gene Cell Line: Molecular Constructs

FIG. 3 illustrates the alignment of optimized synthetic (SEQ ID NO:1) and native beta-Klotho genes.

FIG. 4 illustrates the response of HEK293 cells transfected with the optimized synthetic beta-Klotho gene treated with increasing concentrations of human FGF21.

FIG. 5 illustrates the comparison of the response of HEK293 cells and Jurkat cells transfected with the optimized synthetic and native beta-Klotho genes respectively.

FIG. 6 illustrates the effect of normal human serum on the response of HEK293 cells transfected with the optimized synthetic native beta-Klotho gene.

FIG. 7 illustrates the effect of normal serum on HEK293 cells transfected with the firefly luciferase reporter-gene under the control of either the chimeric gal4-Elk-1 transcription factor as described in the invention or a 6-fold tandem repeat of the canonical native Elk-1 recognition sequence (Sequence ID NO.: 8, FIG. 8). As shown in FIG. 7 treatment of HEK293 cells with DMEM culture medium containing 10% normal serum increased the activity of firefly luciferase reporter-gene under the control of 6-fold tandem repeat of the canonical Elk-1 recognition sequence by approximately 32% relative to cells in DMEM culture medium alone. In contrast, HEK293 cells transfected with the firefly luciferase reporter-gene under the control of the chimeric gal4-Elk-exhibited only a 9.5% increase in the activity of firefly luciferase in the presence of DMEM containing 10% normal serum relative to cells in DMEM culture medium alone.

FIG. 8 illustrates a 6-fold tandem repeat of the canonical native Elk-1 recognition sequence.

Table 1 shows the comparison of the response of cells transfected with the optimized synthetic and native beta-Klotho genes in the freeze, thaw and use format,

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

The present inventors provide a reporter cell line and methods of using the same for detecting/quantitating FGF21 activity in a test sample. The inventors further provide a method for detecting/quantitating antibodies against FGF21. One method includes testing the capacity of an antibody to neutralize the FGF21 activity.

In order to develop a reporter-gene assay specific for FGF21 and without interference from other growth factors present in human serum that use the same signal transduction pathway as FGF21, the inventors have co-transfected human cells including the T-cell line Jurkat cells (ATCC® TIB-152) and the embryonic kidney cell line HEK293 cells (ATCC® CRL-1573) with a firefly luciferase (FL) reporter-gene construct regulated by a chimeric transcription factor consisting of the trans-activation domain of Elk-1 fused to a DNA binding domain that does not exist in mammalian cells, such as that of Gal4. The cells are co-transfected with an expression factor encoding the chimeric transcription factor such that only the chimeric transcription factor will bind to the sequence regulating the reporter gene, such as the up-stream activation sequence (UAS) of gal4. Native endogenous Elk-1 is produced and acts in an identical manner as in wild type cells but will not bind to or activate the chimeric promoter regulating the FL reporter-gene that is regulated exclusively by the chimeric transcription factor. The cells were also co-transfected with expression vectors encoding the FGFR1c receptor chain, and the beta-Klotho co-receptor and the gene encoding Renilla luciferase under the control of a constitutive promoter. The resultant reporter gene cell lines were used as the basis for the establishment of sensitive and specific assays for FGF21 with an EC50 of 170 and 24 ng/ml for Jurkat cells and HEK293 cells respectively with minimal interference from normal human serum. The FGF21 responsive cells are produced in the freeze, thaw and use format described in U.S. Pat. No. 9,188,580.

The inventors further investigated how to further improve the reporter cell. In particular, the inventors investigated how to increase the sensitivity for the detection of FGF21 activity while exhibiting minimal interference from normal human serum.

### Definitions

### Vector

The term “vector” or vector construct” refers to a DNA molecule used as a vehicle to transfer recombinant genetic material into a host cell. The four major types of vectors are plasmids, bacteriophages and other viruses, cosmids, and artificial chromosomes. The vector itself is generally a DNA sequence that consists of an insert (a heterologous nucleic acid sequence, transgene) and a larger sequence that serves as the “backbone” of the vector. The purpose of a vector which transfers genetic information to the host is typically to isolate, multiply, or express the insert in the target cell. Vectors called expression vectors (expression constructs) are specifically adapted for the expression of the heterologous sequences in the target cell, and generally have a promoter sequence that drives expression of the heterologous sequences. The choice of vector employed in embodiments of the present invention depends on the specific application of the vector encoding the polypeptides or polynucleotide.

### Operatively Linked

The term “operatively linked” refers to the connection of elements being a part of a functional unit such as a gene or an open reading frame. Accordingly, by operatively linking a promoter to a nucleic acid sequence encoding a polypeptide (an open reading frame, ORF) the two elements becomes part of the functional unit—a gene. The linking of the expression control sequence (promoter) to the nucleic acid sequence enables the transcription of the nucleic acid sequence directed by the promoter. By operatively linking two heterologous nucleic acid sequences encoding a polypeptide the sequences becomes part of the functional unit—an open reading frame encoding a fusion protein comprising the amino acid sequences encoding by the heterologous nucleic acid sequences. By operatively linking two amino acids sequences, the sequences become part of the same functional unit—a polypeptide. Operatively linking two heterologous amino acid sequences generates a hybrid (fusion) polypeptide.

A first aspect of the present invention concerns a mammalian cell line comprising
(i) a first heterologous polynucleotide comprising a heterologous cis-acting regulatory sequence operably linked to a downstream promoter sequence, wherein said promoter is operably linked to an open reading frame encoding a first reporter protein,
(ii) a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and
(iii) a cell surface bound heterodimeric receptor protein comprising a tyrosine kinase FGFR1c and a beta-Klotho protein.

The mammalian cell line of the present invention (also referred to as the reporter cell line) is useful for assaying for the presence of FGF21 activity in a test sample. The reporter cell line may also be used for testing (such as screening or validating) the activity and potency of analogues of FGF21. Likewise, the mammalian cell line of the present invention may be used for testing (such as screening or validating) antibodies for the capacity (and potency) to neutralize the activity of FGF21.

In order to minimize the background activity of the reporter gene in the mammalian cell, the inventors preferably use a DNA binding domain and a cognate cis-acting regulatory sequence, which is not present in the genetic background of the mammalian cell to be used as host for the reporter system. Useful DNA binding domain/cis-acting regulatory sequence is typically found in organism remotely related to mammal, such as yeast or bacteria. Thus, in one embodiment of the present invention, the heterologous DNA binding domain and its cognate cis-acting regulatory sequence is yeast or bacterial of origin.

The recruitment of the chimeric transcription factor comprising trans-activation domain of Elk-1 fused to a heterologous DNA binding domain and binding to a cognate cis-acting regulatory sequence upstream of the promoter directing the reporter gene encoding the first report protein will stimulate the formation of the transcription complex om the promoter and thus the expression of the first reporter protein. The stimulated expression of the reporter is mediated by the FGF21 signal transduction pathway.

In a preferred embodiment, the cis-acting regulatory sequence is an upstream activation sequence (UAS) and said heterologous DNA binding domain is a DNA binding domain capable of bind to said upstream activation sequence.

In an even more preferred embodiment, the cis-acting regulatory sequence is a galactose-responsive upstream activation sequence (UAS_{G}) and said heterologous DNA binding domain is the DNA binding domain galactose-responsive transcription factor GAL4 (GAL4DB).

In another embodiment, the cis-acting regulatory sequence is the DNA binding site of LexA and said heterologous DNA binding domain is a DNA binding domain of repressor LexA protein.

Other useful systems include the TetR/TetO system and the Cumate/CuO system.

The cell used as host for establishing the reporter cell line of the invention may endogenously express a beta-Klotho protein, the protein that forms part of the cell surface bound heterodimeric receptor protein comprising a tyrosine kinase FGFR1c and a beta-Klotho protein.

However, in a preferred embodiment, an expression vector encoding a beta-Klotho protein and adapted to expressing the same is introduced into the mammalian cell. Thus in one embodiment, the cell comprises a heterologous polynucleotide comprising promoter operably linked to a down-stream open reading frame encoding a beta-Klotho protein and from which said beta-Klotho protein is expressed. The heterologous polynucleotide is typically an expression vector. The promoter operably linked to a down-stream open reading frame encoding a beta-Klotho protein is typically a promoter providing constitutive expression of beta-Klotho protein in the cell line. Examples of promoters useful for directing the expression of beta-Klotho protein in the cell line includes but are not limited to constitutively active promoters selected from the list consisting of cytomegalovirus (CMV) early enhancer/promoter, SV40 promoter, UBC promoter, PGK promoter, human β-actin (hACTB), human elongation factor-1α (hEF-1α), Thymidine Kinase (TK) promoter and cytomegalovirus early enhancer/chicken β-actin (CAG) promoters.

The inventors have found that the EC50 of the reporter system was significantly improved by the introduction of an expression construct encoding a beta-Klotho protein, where coding sequences (the ORF) has been optimized for expression in the mammalian host cell as compared to an expression construct comprising a native beta-Klotho protein.

Accordingly, in one embodiment of the present invention, the open reading frame encoding a beta-Klotho protein has been codon-optimized for expression in said cell. In a particular embodiment, the open reading frame comprises or consist of the sequence set forth in SEQ ID NO: 1 or a sequence having at least 75% sequence identity to SEQ ID NO: 1, such as 79% sequence identity to SEQ ID NO: 1, for example 85% sequence identity to SEQ ID NO: 1, such as 90% sequence identity to SEQ ID NO: 1, such as 95% sequence identity to SEQ ID NO: 1, for example 97% sequence identity to SEQ ID NO: 1, such as 98% sequence identity to SEQ ID NO: 1, for example 99% sequence identity to SEQ ID NO: 1.

In a preferred embodiment, the mammalian cell comprises a heterologous polynucleotide comprising promoter operably linked to a down-stream open reading frame encoding a beta-Klotho protein and from which said beta-Klotho protein is expressed, where the sequence of said open reading frame comprises or consist of the sequence set forth in SEQ ID NO: 1.

The reporter system may use any reporter gene suitable for the application. Typically, the system will use a reporter that is easy to establish in the test lab and which provides great sensitivity. Typically, the reporter gene encodes a reporter protein, which is an enzyme or a fluorescent protein.

In one embodiment, the reporter gene (the first reporter gene) encodes an enzyme. In a preferred embodiment, the reporter (the first reporter) is a luciferase, such as firefly luciferase or Renilla luciferase.

In another embodiment, the reporter gene (the first reporter gene) encodes a fluorescent protein and the first reporter protein is a fluorescent protein. Useful fluorescent protein includes green fluorescent protein (GFP) and related fluorescent protein, e.g. enhanced green fluorescent protein (EGFP), yellow fluorescent protein (YFP), blue fluorescent protein (BFP) and variant there of displaying a different excitation/emission spectra.

The inventors have found that it is useful to introduce a second reporter gene in the reporter cell line of the present invention (the mammalian cell). The second reporter gene is typically introduced in the form of a polynucleotide comprising promoter operably linked an open reading frame encoding the second reporter protein. The promoter directing the expression of the second reporter is typically constitutively active in the mammalian host cell used to establish the reporter cell line of the present invention. Useful constitutively active promoters include but at not limited to cytomegalovirus (CMV) early enhancer/promoter, SV40 promoter, UBC promoter, PGK promoter, human β-actin (hACTB), human elongation factor-1α (hEF-1α), Thymidine Kinase (TK) promoter and cytomegalovirus early enhancer/chicken β-actin (CAG) promoters.

The introduction of the second reporter protein in the mammalian cell reduces the inter-sample variation. The application of the second reporter protein provides means for compensating for inter-sample variation in cell densities due to loss of cells or variation in number of cells seeded.

The advantage of using the constitutive expression of any luciferase is that the results are not influenced by loss of the reporter gene cells, nor are the results influenced by serum matrix effects and provides a constant readily detectable level of luciferase activity. All of these can be compensated for by the normalization obtained through the use of the measurement of the constitutive expression of the other (second) luciferase. None of these advantages can be obtained with the use of a single reporter gene procedure which does not use such normalization.

Where a first and a second reporter protein is present, the reporters a typically selected such that same means for detection is used. For example, if the first reporter is a fluorescent protein such as a enhanced green fluorescent protein (EGFP), the second reporter protein is typically another species of this class of fluorescent protein, displays a different excitation/emission spectra.

In a preferred embodiment, the first reporter protein is a luciferase and the second reporter (constitutively expressed) is also a luciferase with the proviso that the luciferases are not the same. In this way, the activity of both luciferases is readily detectable and can be read sequentially in the same well of an assay plate. For example, when the reporter gene construct produces firefly luciferase (first reporter protein), the constitutive production may be of a second luciferase (second reporter protein), e.g., Renilla luciferase. The activity of the first luciferase normalized relative to the activity of the second luciferase is described in US 2011/0189658. When conducting the assay, after the first reporter gene luciferase is measured, then a reagent is added to quench that specific luciferase so that any following reading will just read the luciferase from the constitutive construct, which then may be used for the purpose of normalization, as will be described in more detail in Example 1.

The advantage of using the constitutive expression of any luciferase is that the results are not influenced by loss of the reporter gene cells, nor are the results influenced by serum matrix effects and provides a constant readily detectable level of luciferase activity. All of these can be compensated for by the normalization obtained through the use of the measurement of the constitutive expression of the other (second) luciferase. None of these advantages can be obtained with the use of a single reporter gene procedure which does not use such normalization.

The cell line used as host for establishing a may be any mammalian cell line comprising an intact FGF21 signal transduction pathway, thus where binding of FGF21 to the FGFR1c receptor in the presence of the beta-Klotho co-receptor leads to activation of the ras/raf MAP kinase signaling cascade, activation of ERK1/2 and phosphorylation and activation of the Elk-1 and SRF. Alternatively, it should be possible to restore the signalling pathway, e.g. by heterologous expression of the beta-Klotho co-receptor, where the receptor is absent and the underlying deficiency in the signalling pathway. Preferably the mammalian cell of the present invention is based on a mammalian host cell, where the FGF21 signal transduction pathway is intact.

In one embodiment, of the present invention, the cell line is selected from the group consisting of HEK293, Jurkat, K652 and U937. In a preferred embodiment, the cell line is HEK293.

The present invention provides a substantial improvement in the freeze, thaw and use format as described in U.S. Pat. No. 9,188,580 applied previously to FGF21 responsive cells in order to provide an improved sensitivity, while retaining minimal interference from normal human serum. As disclosed herein with Example 1, the improved sensitivity manifests itself in a substantially improved EC50 and a substantially improved EC50 and LLOQ (Lower Limit of Quantification).

These features are obtained by means of the present invention in which HEK293 cells were co transfected with the same reporter-gene construct, chimeric Elk-1 transcription factor, and FGFR1c receptor chain as used previously but with an optimized synthetic beta-Klotho gene with 79% homology to the native gene, and the gene encoding Renilla luciferase under the control of a constitutive promoter. The synthetic beta-Klotho gene has been designed to use the codons most frequently employed statistically in order to optimize translation and hence increase the efficiency with which the beta-Klotho co-receptor facilitates the binding of FGF21 to its receptor and activation of the signal transduction pathway. The resultant cell line is markedly more sensitive than the FGF21 responsive Jurkat or HEK293 cell lines previously established using the same cloning strategy but employing the native beta-Klotho gene.

As discussed herein, the mammalian cell line of the present invention is useful for assaying for the presence of FGF21 activity in a test sample. In another aspect, the reporter cell line of the present invention is used for testing (such as screening or validating) the activity and potency of analogues of FGF21. Likewise, the reporter cell line of the present invention may be used for testing (such as screening or validating) antibodies for the capacity (and potency) to neutralize the activity of FGF21.

Accordingly, one aspect of the present invention provides a method for detecting and optionally quantitating FGF21 activity in a test sample, said method comprising the steps of
(i) providing a test sample,
(ii) contacting said test sample with the cell line according to any one of the preceding claims,
(iii) determining the activity of the first reporter protein in said cell line.

The method may be used to detect/quantitate FGF21 activity or the activity/potency of analogous of FGF21.

In a preferred embodiment, the method uses a second reporter protein as means for compensating for e.g. inter-sample variation.

Thus, in one embodiment, the cell line expresses a second reporter protein, said method further comprising
(iv) determining the activity of the first reporter protein in said cell line,
(v) providing the ratio between the activity of the first reporter protein and the second reporter protein.

A further aspect concerns, a method for detecting and optionally quantitating neutralizing antibodies against FGF21 present in a test sample, said method comprising the steps of
(i) providing a test sample comprising,
(ii) providing a first and a second cell sample, wherein said cells samples comprises the cell line according to any one of the preceding claims
(iii) contacting the first cell sample with the test sample and subsequently with FGF21,
(iv) contacting the second cell sample with FGF21,
(iv) determining the activity of the first reporter protein in the cells of the first cell sample and determining the activity of the first reporter protein in the cells of the second cell sample,
(v) provide the ratio between the reporter activity in first and a second cell sample, where a ratio (first/second) lower than one is indicative for the presence of antibodies against FGF21 in said sample.

In a preferred embodiment, the method uses a second reporter protein as means for compensating for e.g. inter-sample variation.

Thus, in one embodiment, the method for detecting and optionally quantitating antibodies against FGF21 present in a test sample comprises the steps of
(i) providing a test sample comprising,
(ii) providing a first and a second cell sample, wherein said cells samples comprises the cell line according to any one of the preceding claims, wherein said cell line expresses a second reporter protein
(iii) contacting the first cell sample with the test sample and subsequently with FGF21,
(iv) contacting the second cell sample with FGF21,
(iv) determining the activity of the first and second reporter protein in the cells of the first cell sample and determining the activity of the first and second reporter protein in the cells of the second cell sample,
(v) provide a first ratio between the reporter activity of the first and second reporter protein in the first cell sample, provide a second ratio between the reporter activity of the first and second reporter protein in the second cell sample
(vi) provide a third ratio between first and second ratio of step (v), where a ratio (first/second) lower than one is indicative for the presence of antibodies against FGF21 in said sample.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended to be optionally substitutable with the terms “consisting of”, “consist of” and “consist of”, respectively, in every instance. The invention will be more fully understood by reference to the following examples. They should not, however, be construed as limiting the scope of the invention. All literature citations are incorporated by reference.

The invention will hereafter be described by way of the following non-limiting items.
Item 1. A mammalian cell line comprising
   (i) a first heterologous polynucleotide comprising a heterologous cis-acting regulatory sequence operably linked to a downstream promoter sequence, wherein said promoter is operably linked to an open reading frame encoding a first reporter protein,
   (ii) a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and
   (iii) a cell surface bound heterodimeric receptor protein comprising a tyrosine kinase FGFR1c and a beta-Klotho protein.
Item 2. The mammalian cell according to claim 1, wherein said heterologous DNA binding domain and its cognate cis-acting regulatory sequence is yeast or bacterial of origin.
Item 3. The mammalian cell according to items 1 or 2, wherein said cis-acting regulatory sequence is an upstream activation sequence (UAS) and said heterologous DNA binding domain is a DNA binding domain capable of bind to said upstream activation sequence.
Item 4. The mammalian cell according any one of the preceding items, wherein said cis-acting regulatory sequence is a galactose-responsive upstream activation sequence (UAS_{G}) and said heterologous DNA binding domain is the DNA binding domain galactose-responsive transcription factor GAL4 (GAL4DB).
Item 5. The mammalian cell according any one of the preceding items, wherein said cis-acting regulatory sequence is the DNA binding site of LexA and said heterologous DNA binding domain is a DNA binding domain of repressor LexA protein.
Item 6. The mammalian cell according any one of the preceding items, wherein said cell comprises a heterologous polynucleotide comprising promoter operably linked to a down-stream open reading frame encoding a beta-Klotho protein and from which said beta-Klotho protein is expressed.
Item 7. The mammalian cell according item 6, wherein said open reading frame encoding a beta-Klotho protein has been codon-optimized for expression in said cell.
Item 8. The mammalian cell according any one of items 6 or 7, wherein said open reading frame comprises or consist of the sequence set forth in SEQ ID NO 1 or a sequence having at least 75% sequence identity to SEQ ID NO 1, such as 79% sequence identity to SEQ ID NO 1, for example 85% sequence identity to SEQ ID NO 1, such as 90% sequence identity to SEQ ID NO 1, such as 95% sequence identity to SEQ ID NO 1, for example 97% sequence identity to SEQ ID NO 1, such as 98% sequence identity to SEQ ID NO 1, for example 99% sequence identity to SEQ ID NO 1.
Item 9. The mammalian cell according any one of the preceding items, wherein said first reporter protein is an enzyme, such as a luciferase.
Item 10. The mammalian cell according any one of the preceding items, wherein said first reporter protein is a fluorescent protein.
Item 11. The mammalian cell according any one of the preceding items, wherein said cell further expresses a second reporter protein.
Item 12. The mammalian cell according any one of the preceding items, wherein said second reporter protein is expressed from a constitutive promoter.
Item 13. The cell line according to any of the preceding items, wherein said cell line is selected from the group consisting of HEK293, Jurkat, K652 and U937.
Item 14. A method for detecting and optionally quantitating FGF21 activity in a test sample, said method comprising the steps of
   (i) providing a test sample,
   (ii) contacting said test sample with the cell line according to any one of the preceding items,
   (iii) determining the activity of the first reporter protein in said cell line.
Item 15. The method of item 14, wherein said cell line expresses a second reporter protein, said method further comprising
   (iv) determining the activity of the first reporter protein in said cell line,
   (v) providing the ratio between the activity of the first reporter protein and the second reporter protein.
Item 16. A method for detecting and optionally quantitating neutralizing antibodies against FGF21 present in a test sample, said method comprising the steps of
   (i) providing a test sample comprising,
   (ii) providing a first and a second cell sample, wherein said cells samples comprises the cell line according to any one of the preceding items
   (iii) contacting the first cell sample with the test sample and subsequently with FGF21,
   (iv) contacting the second cell sample with FGF21,
   (iv) determining the activity of the first reporter protein in the cells of the first cell sample and determining the activity of the first reporter protein in the cells of the second cell sample,
   (v) provide the ratio between the reporter activity in first and a second cell sample, where a ratio (first/second) lower than one is indicative for the presence of antibodies against FGF21 in said sample.
Item 17. A method for detecting and optionally quantitating antibodies against FGF21 present in a test sample, said method comprising the steps of
   (i) providing a test sample comprising,
   (ii) providing a first and a second cell sample, wherein said cells samples comprises the cell line according to any one of the preceding items, wherein said cell line expresses a second reporter protein
   (iii) contacting the first cell sample with the test sample and subsequently with FGF21,
   (iv) contacting the second cell sample with FGF21,
   (iv) determining the activity of the first and second reporter protein in the cells of the first cell sample and determining the activity of the first and second reporter protein in the cells of the second cell sample,
   (v) provide a first ratio between the reporter activity of the first and second reporter protein in the first cell sample, provide a second ratio between the reporter activity of the first and second reporter protein in the second cell sample
   (vi) provide a third ratio between first and second ratio of step (v), where a ratio (first/second) lower than one is indicative for the presence of antibodies against FGF21 in said sample.

The sequences set forth in SEQ ID NO: 2 to 8 relates to various polynucleotides useful for the preparation of the mammalian cell line of the present invention.

### EXAMPLES

### Example 1

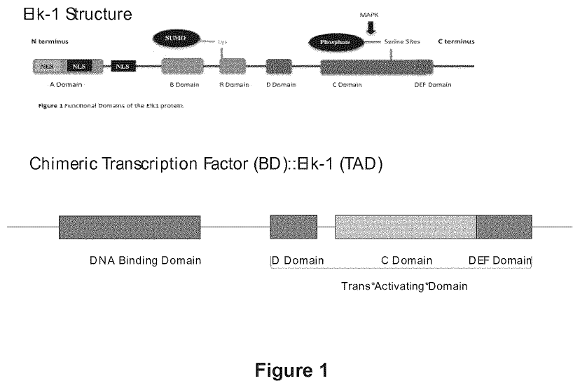

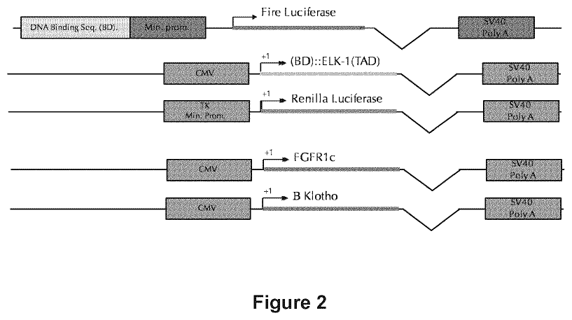

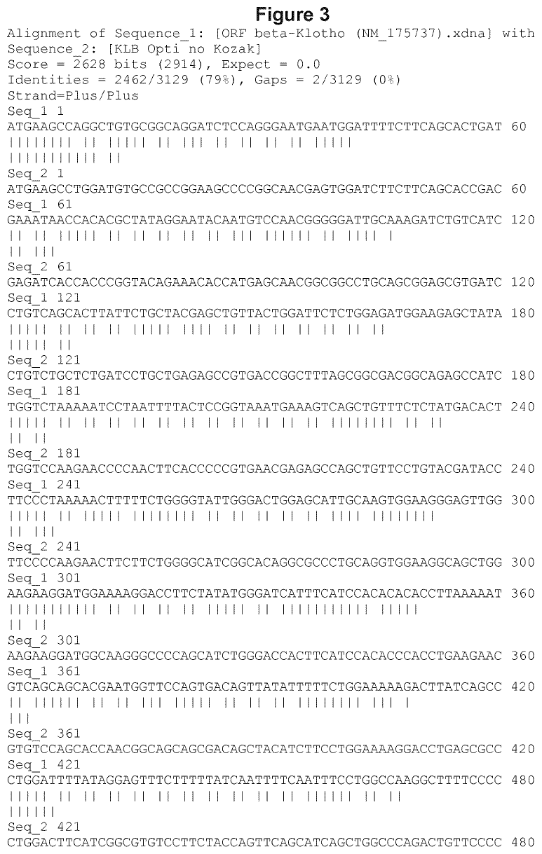

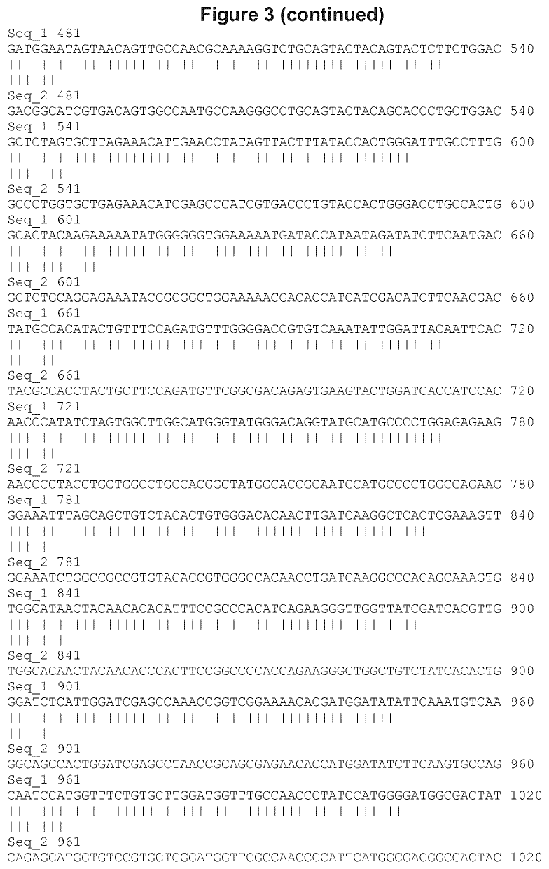

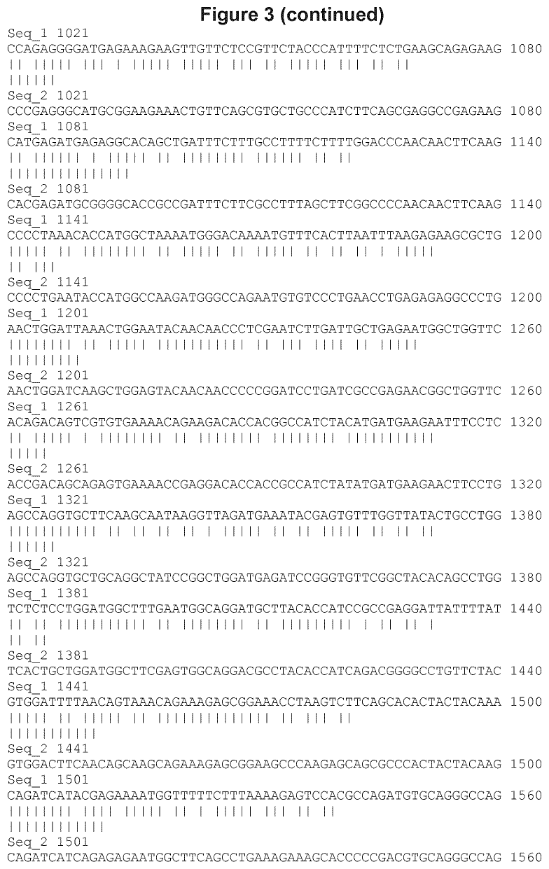

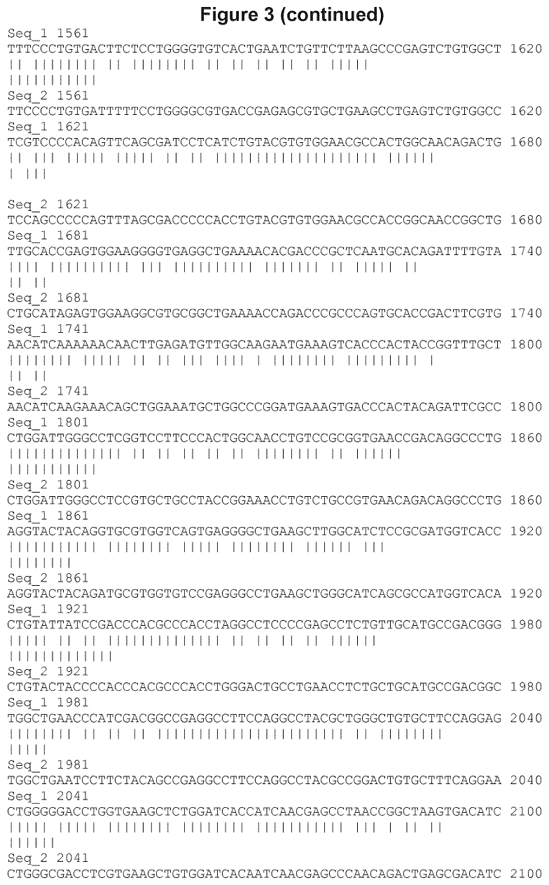

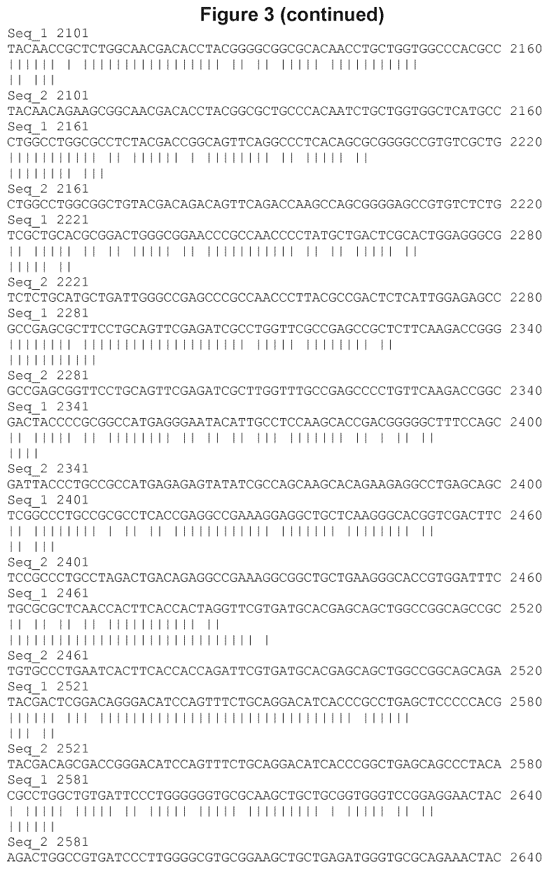

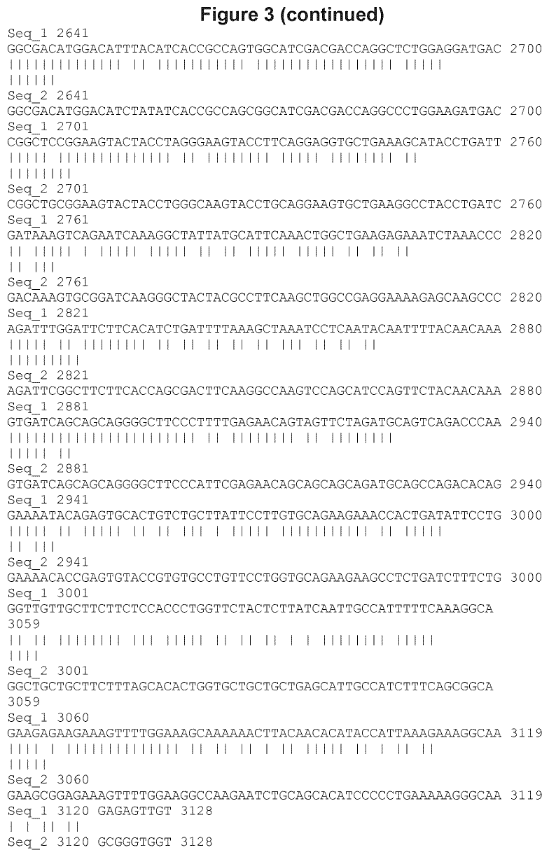

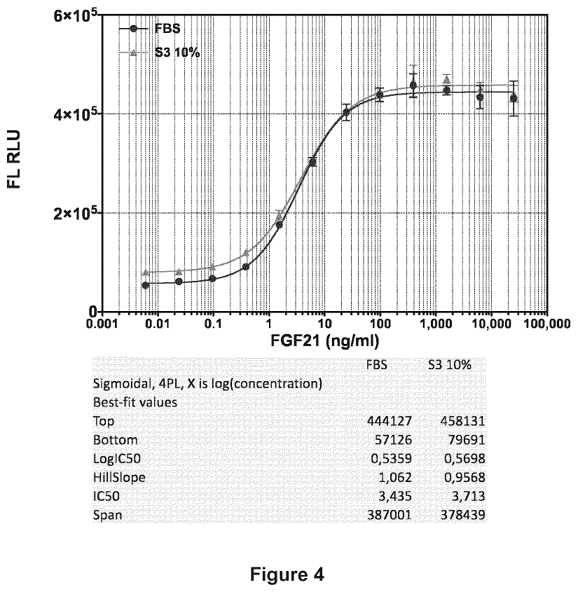

The human embryonic kidney cell line HEK293 (ATCC® CRL-1573) was co-transfected sequentially, using the FuGENE HD transfection reagent (Promega Catalogue No E2311), with a firefly luciferase (FL) reporter-gene construct regulated by a chimeric transcription factor consisting of the trans-activation domain of Elk-1 (nt 919 to nt 1275) fused to the Gal4 DNA binding domain (nt 1 to nt 390) as shown in FIG. 1. Since the gal4 DNA binding domain does not exist in mammalian cells, only the chimeric transcription factor will bind to the up-stream activation sequence (UAS) of gal4 regulating transcription of the FL reporter gene. The cells were also co-transfected with the gene encoding Renilla luciferase under the control of a constitutive promoter used to normalize FGF21 induced FL activity, and the FGFR1c receptor chain together with an optimized synthetic beta-Klotho gene (FIG. 2). The optimized synthetic beta-Klotho gene exhibits 79% homology with the native gene (FIG. 3). The nucleotide sequence of the optimized synthetic beta-Klotho gene is set forth in SEQ ID NO: 1. Stable clones were isolated and characterized for FGF21 responsiveness in the presence of increasing concentrations of human FGF21 and then sub-cloned. Suitable sub-clones were isolated, characterized and propagated, giving rise to a FGF21 responsive cell line with enhanced sensitivity relative to cell lines transfected with the native beta-Klotho gene. Thus, the resultant reporter gene cell line was found to be highly sensitive with an EC50 of approximately 3.0 ng/ml when treated with increasing concentrations of human FGF21 (FIG. 4). Human serum from a normal donor at a 1/10 final dilution did not significantly affect the response of the cells to treatment with increasing concentrations of human FGF21 (FIG. 4).

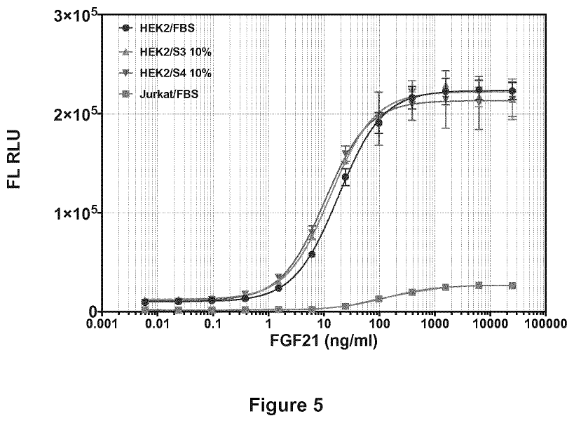

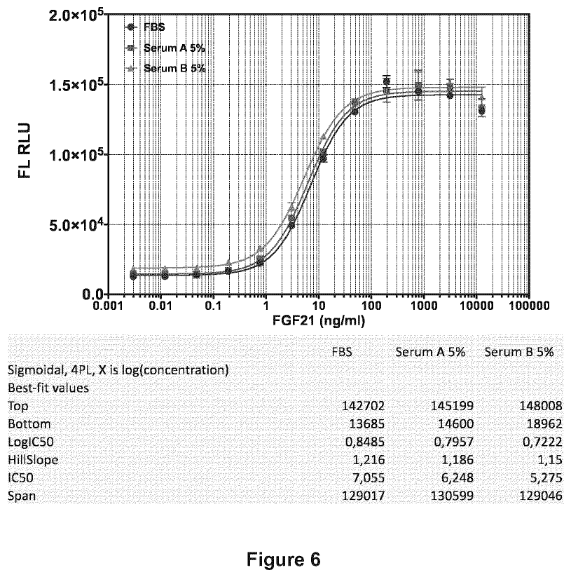

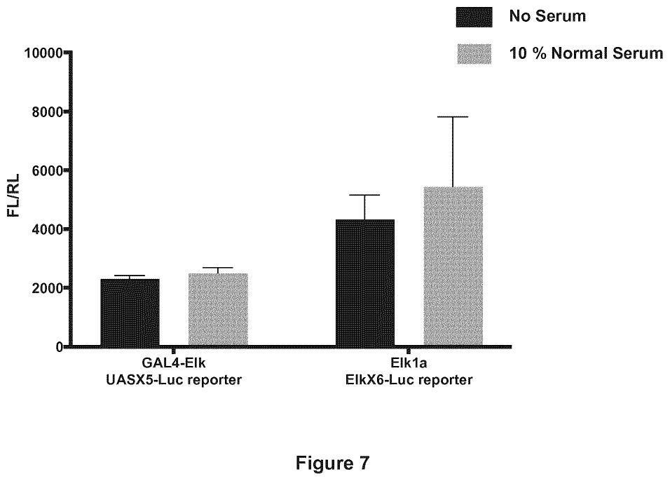

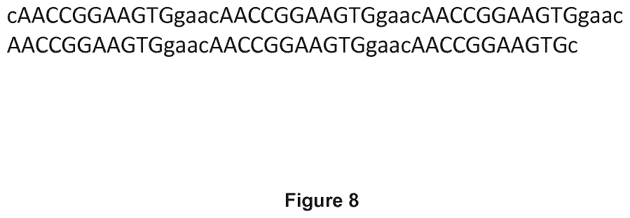

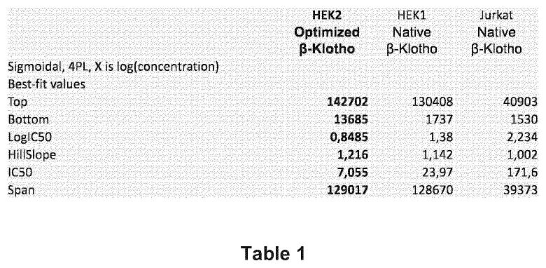

When the FGF21 responsive HEK293 cell line with the optimized synthetic beta-Klotho gene was used in the freeze, thaw, and use, format described in U.S. Pat. No. 9,188,580 and treated with increasing concentrations of human FGF21 the response of the cells expressed as RLU of the firefly luciferase activity, was clearly superior to that observed with the FGF21 responsive Jurkat cell line transfected with the native beta-Klotho gene (FIG. 5). The FGF21 responsive HEK293 cell line with the optimized synthetic beta-Klotho gene used in the freeze, thaw, and use, format described in U.S. Pat. No. 9,188,580 exhibited an EC50 of approximately 7.0 ng/ml compared to an EC50 of approximately 24 ng/ml and 170 ng/ml for HEK293 cells and Jurkat cells respectively transfected with the native beta-Klotho gene and used in the freeze, thaw and use format described in U.S. Pat. No. 9,188,580 (Table 1). The HEK293 cell line with the optimized synthetic beta-Klotho gene used in the freeze, thaw and use format described in U.S. Pat. No. 9,188,580 exhibited no significant interference from normal human serum (FIG. 6).

### REFERENCES

W Y, S., & Leung, P. S., Med. Res. Rev.; 4:672-705, 2016
Kliewer S. A, Mangelsdorf D. J. Am. J. Clin. Nutr. 91:254-257, 2010
Fisher, F. M. et al., Diabetes, 59:2781-2789.
Wang Y, Sun Z, Ageing Research Reviews 8:43-51, 2009
Wang H., et al, Cancer Res. 57:1750-1757, 1997

1. A mammalian cell line comprising
(i) a first heterologous polynucleotide comprising a heterologous cis-acting regulatory sequence operably linked to a downstream promoter sequence, wherein said promoter is operably linked to an open reading frame encoding a first reporter protein,
(ii) a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and
(iii) a cell surface bound heterodimeric receptor protein comprising a tyrosine kinase FGFR1c and a beta-Klotho protein,
wherein said cell comprises a heterologous polynucleotide comprising promoter operably linked to a down-stream open reading frame encoding a beta-Klotho protein and from which the beta-Klotho protein is expressed, and
wherein said open reading frame comprises or consist of the sequence set forth in SEQ ID NO.: 1.

2. A mammalian cell line according to claim 1, wherein the DNA binding domain does not exist in mammalian cells.

3. The mammalian cell according to claim 1, wherein said heterologous DNA binding domain and its cognate cis-acting regulatory sequence is yeast or bacterial of origin.

4. The mammalian cell according to claim 1, wherein said cis-acting regulatory sequence is an upstream activation sequence (UAS) and said heterologous DNA binding domain is a DNA binding domain capable of bind to said upstream activation sequence.

5. The mammalian cell according to claim 1, wherein said cis-acting regulatory sequence is a galactose-responsive upstream activation sequence (UASG) and said heterologous DNA binding domain is the DNA binding domain galactose-responsive transcription factor GAL4 (GAL4DB).

6. The mammalian cell according to claim 1, wherein said cis-acting regulatory sequence is the DNA binding site of LexA and said heterologous DNA binding domain is a DNA binding domain of repressor LexA protein.

7. The mammalian cell according to claim 1, wherein said open reading frame encoding a beta-Klotho protein has been codon-optimized for expression in said cell.

8. The mammalian cell according to claim 1, wherein said first reporter protein is an enzyme.

9. The mammalian cell according to claim 1, wherein said first reporter protein is a luciferase.

10. The mammalian cell according to claim 1, wherein said cell further expresses a second reporter protein.

11. The mammalian cell according to claim 1, wherein said second reporter protein is expressed from a constitutive promoter.

12. The cell line according to claim 1, wherein said cell line is selected from the group consisting of HEK293, Jurkat, K652 and U937.

13. The cell line according to claim 1, wherein said cell line is HEK293.

14. A method for detecting and optionally quantitating FGF21 activity in a test sample, said method comprising the steps of
(i) providing a test sample,
(ii) contacting said test sample with the cell line according to claim 1,
(iii) determining the activity of the first reporter protein in said cell line.

15. The method according to claim 14, wherein detection and optionally quantification of FGF21 activity is essentially without interference of normal human serum.

16. The method according to claim 14, wherein said cell line expresses a second reporter protein, said method further comprising
(iv) determining the activity of the first reporter protein in said cell line,
(v) providing the ratio between the activity of the first reporter protein and the second reporter protein.

17. A method for detecting and optionally quantitating neutralizing antibodies against FGF21 present in a test sample, said method comprising the steps of
(i) providing a test sample comprising,
(ii) providing a first and a second cell sample, wherein said cells samples comprises the cell line according to claim 1,
(iii) contacting the first cell sample with the test sample and subsequently with FGF21,
(iv) contacting the second cell sample with FGF21,
(iv) determining the activity of the first reporter protein in the cells of the first cell sample and determining the activity of the first reporter protein in the cells of the second cell sample,
(v) provide the ratio between the reporter activity in first and a second cell sample, where a ratio (first/second) lower than one is indicative for the presence of antibodies against FGF21 in said sample.

18. The method according to claim 17, wherein detection and optionally quantification of neutralizing antibodies against FGF21 is essentially without interference of normal human serum.

19. The method for detecting and optionally quantitating antibodies against FGF21 present in a test sample according to claim 17, said method comprising the steps of
(i) providing a test sample comprising,
(ii) providing a first and a second cell sample, wherein said cells samples comprises a mammalian cell line comprising a first heterologous polynucleotide comprising a heterologous cis-acting regulatory sequence operably linked to a downstream promoter sequence, wherein said promoter is operably linked to an open reading frame encoding a first reporter protein, a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and a cell surface bound heterodimeric receptor protein comprising a tyrosine kinase FGFR1c and a beta-Klotho protein, wherein said cell comprises a heterologous polynucleotide comprising promoter operably linked to a down-stream open reading frame encoding a beta-Klotho protein and from which the beta-Klotho protein is expressed, and wherein said open reading frame comprises or consist of the sequence set forth in SEQ ID NO.: 1, wherein said cell line expresses a second reporter protein,
(iii) contacting the first cell sample with the test sample and subsequently with FGF21,
(iv) contacting the second cell sample with FGF21,
(iv) determining the activity of the first and second reporter protein in the cells of the first cell sample and determining the activity of the first and second reporter protein in the cells of the second cell sample,
(v) provide a first ratio between the reporter activity of the first and second reporter protein in the first cell sample, provide a second ratio between the reporter activity of the first and second reporter protein in the second cell sample, and
(vi) provide a third ratio between first and second ratio of step (v), where a ratio (first/second) lower than one is indicative for the presence of antibodies against FGF21 in said sample.
